(19) United States
(12) Patent Application Publication
(10) Pub. No.: US 2023/0148890 A1
(43) Pub. Date: May 18, 2023

(54) METHOD FOR INTRINSIC CONTRAST MRI WITH ELECTROPERMANENT MAGNETS

(71) Applicant: Weinberg Medical Physics, Inc., North Bethesda, MD (US)

(72) Inventor: Irving N. WEINBERG, North Bethesda, MD (US)

(21) Appl. No.: 17/987,262

(22) Filed: Nov. 15, 2022

Related U.S. Application Data
(60) Provisional application No. 63/279,524, filed on Nov. 15, 2021.

(51) Int. Cl. A61B 5/05; A61B 5/026

(52) U.S. Cl. CPC A61B 5/05; A61B 5/026

(57) ABSTRACT

A method is provided for detecting tumors and other lesions or structures of interest in parts of human or non-human animals without administering extrinsic contrast materials, using MRI systems that incorporate electropermanent magnets (“EPMs”). The method may include comparing of the spin decay characteristics of the tissues at the quasi-static field and the different quasi-static field to differentiate the types of tissues.

### CROSS-REFERENCE AND PRIORITY CLAIM

This patent application claims priority to U.S. Provisional Patent Application No. 63/279,524, entitled “METHOD FOR INTRINSIC CONTRAST MRI WITH ELECTROPERMANENT MAGNETS,” filed Nov. 15, 2021, the disclosure of which being incorporated herein by reference in its entirety.

### FIELD

Disclosed embodiments are directed to systems and methods for diagnosis, therapy or research involving humans or other animals, in particular, using MRI systems that incorporate electropermanent magnets (“EPMs”).

### BACKGROUND

The term “contrast” is used in this disclosure to describe a difference in measurable properties between one tissue and another, for example an increased signal intensity visible in a magnetic resonance image for one tissue type (e.g., a tumor) as compared to another type (e.g., non-tumor tissue), or increased attenuation visible on an x-ray computed tomography image. The terms “intravenous contrast” and “contrast-enhanced” are used in this disclosure to describe a procedure in which material (e.g., containing gadolinium) is administered to a subject to enhance contrast. The term “intrinsic contrast” is used to describe the situation where tissues demonstrate contrast without the need for intravenous contrast.

Contrast-enhanced magnetic resonance imaging has been shown to be helpful in detecting breast cancers. Intravenous contrast materials can cause unwanted and sometimes fatal hypersensitivity reactions in some women.

Medical facilities that wish to provide contrast-enhanced magnetic resonance imaging to women need to have personnel capable of both administering the contrast and of dealing with potential hypersensitivity reactions. Another disadvantage of intravenous contrast to detect or delineate tumors is that the tumor-to-nontumor contrast only lasts while the contrast material is flowing through the tumor, thereby eliminating the possibility of examining the tissues after removal from the body. Since repeated administration of intra-venous contrast is challenging (at least in part due to increased toxicity), intra-operative examination of tumors is difficult.

Several methods have been employed using conventional MRI systems to detect and delineate breast cancers that utilize intrinsic contrast. These methods include (1) arterial spin labeling, as taught by S L Franklin et al, Feasibility of Velocity-Selective Arterial Spin Labeling in Breast Cancer Patients for Noncontrast-Enhanced Perfusion Imaging, J Magn. Reson. Imaging 54:1282-1291 (2021), in which radiofrequency pulses polarize spins in blood vessels serving the breasts, and (2) fast free-cycling MRI, taught by K J Pine, G R Davis, D J Lurie, Field-cycling NMR relaxometry with spatial selection, Magnetic Resonance in Medicine 63:1698-1702 (2010), and E DiGregorio et al, Use of FCC-NMRD relaxometry for early detection and characterization of ex-vivo murine breast cancer, Nature Scientific Reports 9:4624 (2019), in which the T1 decay of protons is dependent on the cellular composition of tissues. An advantage of fast free-cycling MRI is that tumor-to-nontumor contrast is present even after tissues are removed from the body, as taught by V Bitonto et al, Low-Field NMR Relaxometry for Intraoperative Tumour Margin Assessment in Breast-Conserving Surgery, Cancers 13, 4141 (2021), which is useful in confirming that lumpectomy margins are clear of tumor.

### SUMMARY

Disclosed embodiments describe a method for detecting tumors and other lesions or structures of interest in parts of human or non-human animals without administering extrinsic contrast materials, using MRI systems that incorporate electropermanent magnets (“EPMs”).

According to some embodiments, a method for differentiating between types of tissues of a subject may include actuating at least one electropermanent magnet in the vicinity of a subject to generate a quasi-static field at the tissues, and actuating a pulse sequence that measures the spin decay characteristics of the tissues at that quasi-static field. Additionally the method may include actuating of the at least one electropermanent magnet to generate a subsequent different quasi-static field at the tissues, actuating of a pulse sequence that measures the spin decay characteristics of the tissues at that subsequent different quasi-static field, and comparing of the spin decay characteristics of the tissues at the quasi-static field and the different quasi-static field to differentiate the types of tissues.

In accordance with some embodiments a method for differentiating between types of tissues of a subject may include actuating at least one electropermanent magnet in the vicinity of a subject to generate a quasi-static field in a circulatory organ, actuating the at least one electropermanent magnet to generate a different quasi-static field at the tissues, which are spaced apart from the organ, and actuating of a pulse sequence that measures the spins of blood circulating through tissues and generates an image.

### BRIEF DESCRIPTION OF THE FIGURES

Aspects and features of the disclosed embodiments are described in connection with various figures, in which:

FIG. 1 shows an example of an apparatus according to the disclosed embodiments;

FIG. 2 shows a flowchart of a method of imaging according to the disclosed embodiments; and

FIG. 3 shows another method of imaging according to the disclosed embodiments.

### DETAILED DESCRIPTION

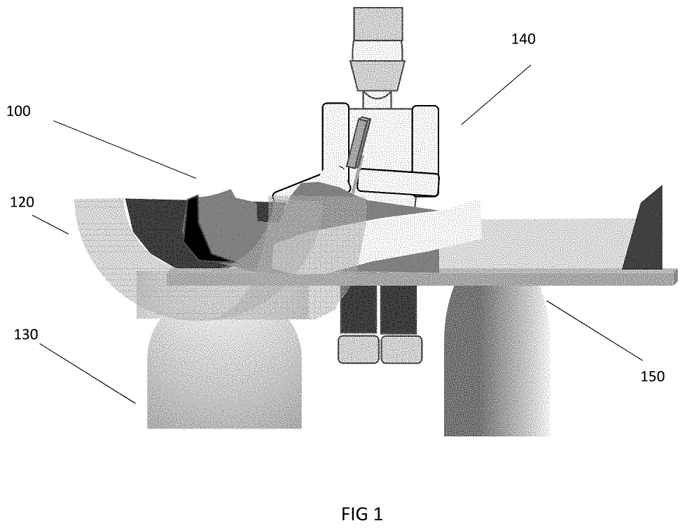

As illustrated in FIG. 1, a human or animal subject 100 is in the field-of-view (FOV) of an assembly of electropermanent magnets 120, the EPMs comprising an imaging system (for example, an MRI). The subject is in the vicinity (for example, within one meter) of the imaging system. In some embodiments, the assembly may be supported by a pillar 130. In some embodiments, subject 100 is supported on a table 150. A practitioner 140 may perform a procedure. Although FIG. 1 illustrates a subject in the vicinity of an EPM array, it is understood that tissues removed from a subject could also be imaged, i.e., by replacing the entire subject with the tissues from the subject.

The apparatus of FIG. 1 employs an array of at least one electropermanent magnet (EPMs) to establish quasi-static magnetic fields and magnetic gradients. Weinberg US 2017/0227617 “Method and apparatus for manipulating electropermanent magnets for magnetic resonance imaging and image guided therapy”, incorporated by reference in its entirety, teaches how to build an MRI with electropermanent magnets, where electropermanent magnets are defined as components containing a magnetizable material and an electrically conductive material, in which the magnetizable material has remanent magnetization after current flows through the electrically conductive material.

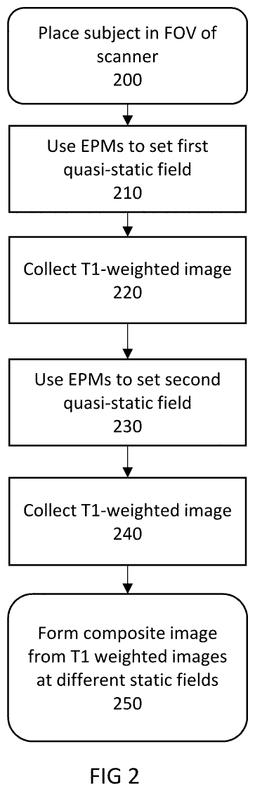

In the method of FIG. 2, the disclosed embodiments take advantage of the dependence of polarization relaxation processes on BO for different materials in the FOV, as taught by Pine, DiGregorio, and Bitonto. The relaxation processes may be manifest in decay time T1, which may differ in different BO field strengths. These differences (in T1 and/or other relaxation processes) allow a computer to differentiate between regions of tumor and non-tumor in a subject, or in tissues removed from a subject. The term quasi-static magnetic field (“BO”) is understood to mean a magnetic field that lasts long enough to polarize protons in the FOV.

In a method according to FIG. 2, in operation 200, tissues of a subject are placed in the FOV of the EPM MRI scanner. The electropermanent magnets set a first quasi-static field (for example a magnetic field lasting more than one second) within the FOV 210. A first MRI pulse sequence is actuated, and measurement of it obtained, for example a T1-weighted pulse sequence 220. The T1-weighted pulse sequence is influenced by the spin decay of the protons as they recover to the ground state. A subsequent different quasi-static field is established 230. A second MRI pulse sequence is actuated and measurement obtained, for example a T1-weighted pulse sequence 240. The results of the first and second MR images are compared to determine the relative T1 decay for tissues in the FOV to differentiate tissue types 250.

Although FIG. 2 illustrates only two quasi-static fields and two MRI pulse sequences, it is understood that more than two of either of these steps may be used.

The term quasi-static field is understood to be a magnetic field lasting long enough to polarize spins in a tissue (e.g., more than one second), but which can be modified at times. This property is achieved through the use of EPM arrays. A conventional superconducting MRI cannot change the static field except by quenching, which is a dangerous procedure. Prior fast field-cycling MRI systems have employed electromagnets (not EPMs) to oppose the static field of a conventional superconducting MRI.

Although FIG. 2 illustrates the use of T1 dependence to differentiate between tissue types (e.g., cancer versus non-cancer), it is understood that other spin relaxation parameters may be assessed through the judicious selection of pulse sequences. For example, quadrupole dips may be measured, as taught by D J Lurie, Quadrupole-Dips Measured by Whole-Body Field-Cycling Relaxometry and Imaging, presented at the 1999 ISMRM conference for use in a fast field-cycling MRI.

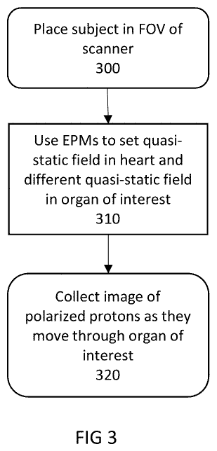

In a method of imaging according to FIG. 3, In operation 300, a subject is placed in the field-of-view (FOV) of the MRI scanner. The electropermanent magnets set a quasi-static field in the region of the heart or other portion of the circulatory system, and a different quasi-static field in an organ of interest (for example, a breast) 310. An MRI pulse sequence is obtained 320, in which spins initially polarized in step 310 and now within the blood flowing through the organ of interest (for example the heart) are imaged. Since cancer tissues often have different perfusion patterns than benign tissues, measuring blood flow or perfusion in tissue by observing the distribution of labeled spins provides information as to whether a tissue is likely to be benign or malignant.

FIG. 3 results in images that show labeled spins, as taught by Franklin. However, in Franklin, the spins were labeled through application of radiofrequency pulses that were selective for specific positions or velocities in the FOV. Since a conventional MRI cannot differentially apply one static field in one location of a body and a different static field at another location, it would not have been possible for Franklin to differentially polarize spins at different location by altering the static field at those locations. However, using and array EPMs it is possible to do so, since the magnetic fields created by some of the EPMs may be oriented (and/or have different magnitudes) than other EPMs in the array.

It is understood that a combination of the methods of FIG. 2 and FIG. 3 may be employed, and that either of these novel methods may be employed with other pulse sequences.

It is understood that the disclosed methods use intrinsic contrast and may be applied multiple times to a subject, unlike methods that use intravenously-injected contrast material. This property would be helpful in surgery or other procedures that benefit from re-examination of a region.

Although the above description mentions the advantage of the method in detecting and delineating a tumor, the method may also be useful in differentiating non-cancer structures from other structures in a body, for example regions in a brain that have more cellularity or whose cells are oriented in a specific direction.

It is understood that the MRI may contain permanent magnets in addition to electropermanent magnets.

Those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments but should instead be defined only in accordance with the following claims and their equivalents.

1. A method for differentiating between types of tissues of a subject, the method comprising:
actuating at least one electropermanent magnet in a vicinity of a subject to generate a first quasi-static field at the tissues,
actuating a pulse sequence and measuring the spin decay characteristics of the tissues at that first quasi-static field, and
actuating at least one second electropermanent magnet to generate a subsequent different quasi-static field at the tissues,
actuating a second pulse sequence and measuring the spin decay characteristics of the tissues at that subsequent different quasi-static field, and
comparing of the spin decay characteristics of the tissues at the first quasi-static field and the different quasi-static field to differentiate the types of tissues.

2. The method of claim 1, wherein the tissues are in the subject.

3. The method of claim 1, wherein the tissues have been removed from the subject.

4. The method of claim 1, wherein the method is repeated multiple times.

5. The method of claim 1, wherein the method is performed using only the intrinsic contrast of the tissues.

6. The method of claim 1, wherein the at least one electropermanent magnet forms part of an MRI system.

7. A method for differentiating between types of tissues of a subject, comprising:
actuating at least one electropermanent magnet in the vicinity of a subject to generate a quasi-static field in a circulatory organ,
actuating at least one second electropermanent magnet to generate a different quasi-static field at the tissues, which are spaced apart from the circulatory organ,
actuating of a pulse sequence and measuring the spins of blood circulating through tissues and generates an image.

8. The method of claim 7, wherein the method is repeated multiple times.

9. The method of claim 7, wherein the method is performed using only the intrinsic contrast of the tissues.

10. The method of claim 7, wherein the at least one electropermanent magnet forms part of an MRI system.
